(19) United States
(12) Patent Application Publication
(10) Pub. No.: US 2011/0229537 A1
(43) Pub. Date: Sep. 22, 2011

(54) ORAL SUPPLEMENT

(75) Inventors: Peter Matravers, Mission Viejo, CA (US); Deborah Hicks, Irvine, CA (US); Sheree Wiener, Laguna Niguel, CA (US); Michele Arth, Golden Valley, MN (US)

(73) Assignee: ARBONNE INTERNATIONAL LLC, Irvine, CA (US)

(21) Appl. No.: 13/050,523

(22) Filed: Mar. 17, 2011

Related U.S. Application Data
(60) Provisional application No. 61/314,944, filed on Mar. 17, 2010.

(51) Int. Cl. A61K 8/97; A61K 8/02; A61Q 19/08

(52) U.S. Cl. 424/401; 424/94.1

(57) ABSTRACT

An oral supplement containing bilberry extract, quercetin, beta-carotene, co-enzyme Q-10, lipoic acid, vitamins, and minerals, which supports skin health, as well as a method of administering the same, alone and in conjunction with a topical skin care composition, is disclosed.

This application claims the benefit of priority from U.S. Provisional Patent Application No. 61/314,944, which was filed on Mar. 17, 2010, the contents of which are incorporated herein in their entirety.

### BACKGROUND

1. Field

This disclosure relates to an oral supplement containing bilberry extract, quercetin, beta-carotene, co-enzyme Q-10, lipoic acid, vitamins, and minerals, as well as to a method of using the same, alone or in conjunction with a topical skin care composition.

2. Related Background Art

The skin, which is the largest (10 pounds) organ in the body, has two major cell types, namely, fibroblasts in the dermal layer and keratinocytes in the epidermis layer. The skin provides the body's first line of defense between the body's interior aggression and harmful environmental insults. Skin deteriorates with age as a natural consequence of prolonged exposure to internal and external factors. Internal deterioration factors include metabolic regenerative slowdown, free radicals, oxidative damage and loss of collagen. External factors include UV radiation, pollution, cigarette smoke, and environmental weathering.

Unfortunately conventional cosmetic products seldom augment the body's metabolism proactively. They only obscure and temporarily mask the signs of aging. It is therefore desirable to have a skin care method and composition which not only reduces the symptoms of deterioration but also treats the underlying causes.

### SUMMARY

The present disclosure is directed to an oral supplement comprising: (a) bilberry extract, (b) quercetin, (c) beta-carotene, (d) co-enzyme Q-10, (e) lipoic acid, (f) vitamin A, (g) vitamin B, (h) vitamin C, (i) vitamin D, (j) vitamin E, (k) selenium, (l) zinc, and (m) copper, wherein the oral supplement supports skin health. In a certain embodiment, the oral supplement may also further comprise at least one inactive ingredient which, in some embodiments, is selected from magnesium stearate, silicon dioxide, microcrystalline cellulose, stearic acid, and combinations thereof. The oral supplement may be in the form of a powder-filled capsule.

The present disclosure is further directed to a method of treating skin comprising the step of: administering to a subject an oral supplement formulated to support skin health, wherein the oral supplement comprises an oral supplement comprising: (a) bilberry extract, (b) quercetin, (c) beta-carotene, (d) co-enzyme Q-10, (e) lipoic acid, (f) vitamin A, (g) vitamin B, (h) vitamin C, (i) vitamin D, (j) vitamin E, (k) selenium, (l) zinc, and (m) copper. In a preferred embodiment, the oral supplement is administered at least once a day. In a preferred embodiment, the oral supplement is administered in the form of at least one powder-filled capsule. In a certain embodiment, the subject suffers from a skin condition selected from the group consisting of wrinkles, fine lines, hyperpigmentation, uneven tone, loss of firmness, creepiness, surface roughness, dark circles, under-eye puffiness, crow's feet, visible sun damage, redness, dryness, irritation, skin sagging, skin slackening, enlarged pores and combinations thereof.

The method of the present disclosure may further comprise the step of: applying a topical skin care composition to the skin of the subject. In a preferred embodiment, the topical skin care composition is applied at least once a day. In a preferred embodiment, the topical skin care composition is applied twice a day.

### DETAILED DESCRIPTION

The present disclosure is directed to an oral supplement which supports skin health. The oral supplement comprises: (a) bilberry extract, (b) quercetin, (c) beta-carotene, (d) co-enzyme Q-10, (e) lipoic acid, (f) vitamin A, (g) vitamin B, (h) vitamin C, (i) vitamin D, (j) vitamin E, (k) selenium, (l) zinc, and (m) copper, which are each selected for the benefits they impart to a subject using the oral supplement. As used herein, “supports skin health” refers to an oral supplement which has a positive effect on the treatment and/or prevention of skin conditions or to an oral supplement which contributes to a positive effect, i.e., increases, speeds up, etc., on the treatment and/or prevention of skin conditions also being treated through the use of a topical skin care composition(s). As further defined herein, “skin conditions” include wrinkles, fine lines, hyperpigmentation, uneven tone, loss of firmness, creepiness, surface roughness, dark circles, under-eye puffiness, crow's feet, visible sun damage, redness, dryness, irritation, skin sagging, skin slackening, enlarged pores and combinations thereof.

The oral supplement of the present disclosure is an excellent source of vitamins and minerals which support a well nourished skin. In addition, the oral supplement supports immune function and consequently healthier skin. The antioxidants in the oral supplement protect cells from free radical damage. The oral supplement, by virtue of its composition, may help support cells, provide broad spectrum antioxidant support to help boost the skin's natural free radical defense system, help to support collagen production/synthesis, help to support skin health and function, help in connective tissue formation, help to promote skin clarity and tone, boost calcium absorption, support eye health, and support energy metabolism.

Bilberry extract, quercetin, beta-carotene, co-enzyme Q-10, and lipoic acid are chosen for at least their antioxidant properties.

Bilberry (Vaccinium myrtillus) or bilberry extract is an antioxidant useful in the maintenance of good health. Bilberry is preferably present in the oral supplement of the present disclosure in an amount ranging from about 6 mg to about 200 mg, more preferably in an amount ranging from about 12 mg to about 100 mg, and most preferably in an amount ranging from about 25 mg to about 50 mg (when the bilberry extract is standardized to 25% anthocyanosides providing 12.5 mg of anthnocyanosides). Preferably bilberry is present in the oral supplement in an amount necessary to provide a daily intake of about 50 mg taking into account dosing frequency.

Quercetin (3,3′,4′5,7-pentahydroxyflavone), a citrus bioflavonoid or flavonoid, is an antioxidant/antioxidant supporter known for its role in cellular health and protection. Quercetin is preferably present in the oral supplement of the present disclosure in an amount ranging from about 2 mg to about 80 mg, more preferably in an amount ranging from about 5 mg to about 40 mg, and most preferably in an amount ranging from about 10 mg to about 20 mg. Preferably quercetin is present in the oral supplement in an amount necessary to provide a daily intake of about 20 mg taking into account dosing frequency. Quercetin dihydrate powder is a preferred form of quercetin for use in the present disclosure.

Beta-carotene is an antioxidant useful in promoting immune function and cellular health and protection. Beta-carotene is preferably present in the oral supplement of the present disclosure in an amount ranging from about 500 IU to about 16000 IU, more preferably in an amount ranging from about 1000 IU to about 8000 IU, and most preferably in an amount ranging from about 2000 IU to about 4000 IU. Preferably beta-carotene is present in the oral supplement in an amount necessary to provide a daily intake of about 4000 IU taking into account dosing frequency. Beta-carotene beadlets are a preferred form of beta-carotene for use in the present disclosure.

Co-enzyme Q-10 (ubidecarenone) is an antioxidant which provides energy at the cellular level. Co-enzyme Q-10 is preferably present in the oral supplement of the present disclosure in an amount ranging from about 3 mg to about 120 mg, more preferably in an amount ranging from about 7 mg to about 60 mg, and most preferably in an amount ranging from about 15 mg to about 30 mg. Preferably co-enzyme Q-10 is present in the oral supplement in an amount necessary to provide a daily intake of about 30 mg taking into account dosing frequency. Co-enzyme Q-10 powder is a preferred form of co-enzyme Q-10 for use in the present disclosure.

Lipoic acid is also a beneficial antioxidant. Lipoic acid is preferably present in the oral supplement of the present disclosure in an amount ranging from about 12 mg to about 400 mg, more preferably in an amount ranging from about 25 mg to about 200 mg, and most preferably in an amount ranging from about 50 mg to about 100 mg. Preferably lipoic acid is present in the oral supplement in an amount necessary to provide a daily intake of about 100 mg taking into account dosing frequency. Alpha-lipoic acid powder is a preferred form of lipoic acid for use in the present disclosure.

Vitamin A, vitamin B, vitamin C, vitamin D, and vitamin E are chosen vitamins for inclusion in the oral supplement.

Vitamin A supports eye health and provides normal immune function. Vitamin A is preferably present in the oral supplement of the present disclosure in an amount ranging from about 125 IU to about 5000 IU, more preferably in an amount ranging from about 250 IU to about 2000 IU, and most preferably in an amount ranging from about 500 IU to about 1000 IU. Preferably vitamin A is present in the oral supplement in an amount necessary to provide a daily intake of about 1000 IU taking into account dosing frequency. Vegetarian vitamin A palmitate is a preferred form of vitamin A for use in the present disclosure.

Vitamin B, especially biotin (cis-hexahydro-2-oxo-1H-thieno [3,4-d]-imidazole-4-valeric acid, a form of vitamin B), plays a role in energy metabolism. Biotin is preferably present in the oral supplement of the present disclosure in an amount ranging from about 37 mcg to about 1200 mcg, more preferably in an amount ranging from about 75 mcg to about 600 mcg, and most preferably in an amount ranging from about 150 mcg to about 300 mcg. Preferably biotin is present in the oral supplement in an amount necessary to provide a daily intake of about 300 mcg taking into account dosing frequency.

Vitamin C (ascorbic acid) is a known antioxidant, which protects the body from free radical damage and helps maintain proper immune function. Vitamin C is preferably present in the oral supplement of the present disclosure in an amount ranging from about 7 mg to about 240 mg, more preferably in an amount ranging from about 15 mg to about 120 mg, and most preferably in an amount ranging from about 30 mg to about 60 mg. Preferably vitamin C is present in the oral supplement in an amount necessary to provide a daily intake of about 60 mg taking into account dosing frequency.

Vitamin D (calcitriol, ergocalciferol) helps to boost calcium absorption. Vitamin D is preferably present in the oral supplement of the present disclosure in an amount ranging from about 62 IU to about 2000 IU, more preferably in an amount ranging from about 125 IU to about 1000 IU, and most preferably in an amount ranging from about 250 IU to about 500 IU. Preferably vitamin D is present in the oral supplement in an amount necessary to provide a daily intake of about 500 IU taking into account dosing frequency. Ergocalciferol is a preferred form of vitamin D for use in the present disclosure.

Vitamin E (mixed tocopherols—alpha, beta, gamma, delta; tocopheryl succinate) is an antioxidant which supports immune function. Vitamin E is preferably present in the oral supplement of the present disclosure in an amount ranging from about 7 IU to about 240 IU, more preferably in an amount ranging from about 15 IU to about 120 IU, and most preferably in an amount ranging from about 30 IU to about 60 IU. Preferably vitamin E is present in the oral supplement in an amount necessary to provide a daily intake of about 60 IU taking into account dosing frequency. Mixed tocopherols is a preferred form of vitamin E for use in the present disclosure.

Selenium, zinc, and copper are chosen for their beneficial properties as minerals.

Zinc (Zn) helps to maintain healthy skin and immune function. Zinc is a known antioxidant Zinc is preferably present in the oral supplement of the present disclosure in an amount ranging from about 1 mg to about 60 mg, more preferably in an amount ranging from about 3 mg to about 30 mg, and most preferably in an amount ranging from about 7.5 mg to about 15 mg. Preferably zinc is present in the oral supplement in an amount necessary to provide a daily intake of about 15 mg taking into account dosing frequency Zinc gluconate is a preferred form of zinc for use in the present disclosure.

Copper (Cu) activates several enzymes involved in the metabolism of amino acids and their metabolites, energy, and the activated form of oxygen, superoxide. Enzyme activation by copper produces physiologically important effects on connective tissue formation, iron metabolism, central nervous system activity, melanin pigment formation, and protection against oxidative stress. Copper is preferably present in the oral supplement of the present disclosure in an amount ranging from about 0.12 mg to about 4 mg, more preferably in an amount ranging from about 0.25 mg to about 2 mg, and most preferably in an amount ranging from about 0.5 mg to about 1 mg. Preferably, copper is present in the oral supplement in an amount necessary to provide a daily intake of about 15 mg taking into account dosing frequency. Copper gluconate is a preferred form of copper for use in the present disclosure.

Selenium (Se) is a constituent of the antioxidant enzyme glutathione peroxidase. Selenium is preferably present in the oral supplement of the present disclosure in an amount ranging from about 6 mcg to about 208 mcg, more preferably in an amount ranging from about 13 mcg to about 104 mcg, and most preferably in an amount ranging from about 26 mcg to about 52 mcg. Preferably selenium is present in the oral supplement in an amount necessary to provide a daily intake of about 52 mcg taking into account dosing frequency. Sodium selenate is a preferred form of selenium for use in the present disclosure.

In an embodiment of the oral supplement of the present disclosure, bilberry extract is present in an amount ranging from about 1.0% to about 7.0%; quercetin is present in an amount ranging from about 1.0% to about 8.0%; beta-carotene is present in an amount ranging from about 1.0% to about 12.0%; co-enzyme Q-10 is present in an amount ranging from about 1.0% to about 16.0%; lipoic acid is present in an amount ranging from about 2.0% to about 40.0%; vitamin A, preferably in the form of vitamin A palmitate, is present in an amount ranging from about 0.1% to about 1.25%; vitamin B, preferably in the form of biotin, is present in an amount ranging from about 0.00375% to about 0.12%; vitamin C, preferably in the form of ascorbic acid, is present in an amount ranging from about 5.0% to about 50.0%; vitamin D, preferably in the form of ergocalciferol, is present in an amount ranging from about 0.1% to about 3.0%; vitamin E, preferably in the form of mixed tocopherols, is present in an amount ranging from about 2.0% to about 50.0%; copper, preferably in the form of copper gluconate, is present in an amount ranging from about 0.08% to about 6.0%; zinc, preferably in the form of zinc gluconate, is present in an amount ranging from about 1.0% to about 48.0%; and selenium, preferably in the form of sodium selenate, is present in an amount ranging from about 0.1% to about 1.16%. Each of the above-noted percentages are expressed by total weight of an oral supplement fill, for example, for use within a capsule, or by total weight of an oral supplement not used as a fill, for example, a pressed powder tablet prior to optional coating.

Table 1 below presents the amount of each component present by percentage and by weight (mg, mcg or IU) in a preferred embodiment of the oral supplement of the present invention.

TABLE 1

| | Percentage per Oral | |
|---|---|---|
| | Supplement | Weight per Oral |
| INGREDIENTS | (approximate %) | Supplement |
| Bilberry fruit extract | 1.0-7.0 | 6 mg-200 mg |
| | | (active) |
| Selenium (as sodium selenate) | 0.1-1.16 | 6 mcg-208 mcg |
| Quercetin dihydrate powder | 1.0-8.0 | 2 mg-80 mg |
| Vitamin E | 2.0-50.0 | 7 IU-240 IU |
| (as mixed tocopherols) | | |
| Co-enzyme Q-10 powder | 1.0-16.0 | 3 mg-120 mg |
| Vitamin B (as biotin) | 0.00375-0.12 | 37 mcg-1200 mcg |
| Vitamin C (as ascorbic acid) | 5.0-50.0 | 7 mg-240 mg |
| Beta carotene beadlet | 1.0-12.0 | 500 IU-16000 IU |
| Vitamin A palmitate, | 0.1-1.25 | 125 IU-5000 IU |
| vegetarian | | |
| Vitamin D2 (as ergocalciferol) | 0.1-3.0 | 62 IU-2000 IU |
| Zinc (as zinc gluconate) | 1.0-48.0 | 1 mg-60 mg |
| Alpha-lipoic acid | 2.0-40.0 | 12 mg-400 mg |
| Copper (as copper gluconate) | 0.08-6.0 | 0.12 mg-4 mg |
| Excipients | QS* | QS |
| Magnesium stearate EF | | |
| Silicon dioxide powder | | |
| Microcrystalline cellulose | | |
| powder | | |
| Stearic acid powder | | |
| TOTAL | 100 | |

*QS = quantity sufficient to equal 100% or to attain the desired total weight.

The above-noted antioxidants, i.e., bilberry extract, quercetin, beta-carotene, co-enzyme Q-10, and lipoic acid, vitamins, i.e., vitamin A, vitamin B, vitamin C, vitamin D, and vitamin E, and minerals, i.e., copper, selenium and zinc, are readily available commercial ingredients and can be purchased from known sources for use in the present disclosure. Additional antioxidants, vitamins and minerals may be suitable for inclusion in the oral supplement of the present disclosure if, when combined, such antioxidants, vitamins and minerals support skin health.

In a certain embodiment of the oral supplement of the disclosure, the oral supplement further comprises at least one inactive ingredient. Preferably, the at least one inactive ingredient is selected from magnesium stearate, silicon dioxide, microcrystalline cellulose, stearic acid, and combinations thereof. One of ordinary skill in the art will readily appreciate that other materials suitable for use as binders, fillers, diluents, and lubricants may be used in the oral supplement of the present disclosure.

The oral supplement of the present disclosure may take any consumer acceptable dosage form, e.g., and without limitation, powder-filled capsule, tablet, softgel, caplet, etc. A preferred form of the oral supplement is a powder-filled capsule. While the desired dosage, i.e., daily dosage, of the oral supplement can be achieved in a singular dosage form, it is possible and sometimes even desirable to split a desired dosage, i.e., daily dosage, between two or more dosage forms.

The present disclosure is further directed to a method of treating skin comprising the step of: administering to a subject an oral supplement formulated to support skin health, wherein the oral supplement comprises (a) bilberry extract, (b) quercetin, (c) beta-carotene, (d) co-enzyme Q-10, (e) lipoic acid, (f) vitamin A, (g) vitamin B, (h) vitamin C, (i) vitamin D, (j) vitamin E, (k) selenium, (l) zinc, and (m) copper. The oral supplement suitable for use in this embodiment of the disclosure is the same as that described above. In a certain embodiment, the oral supplement is administered at least once a day, preferably once or twice a day, though other dosage regimens are also contemplated, i.e., every other day, once a week, etc. As noted above, the oral supplement can be administered in any consumer acceptable dosage form, but is preferably a powder-filled capsule. The desired dosage can be administered in a singular dosage form or split between multiple dosage forms, which can be administered at the same time or at staggered times as desired.

The present method of treating skin is more particularly directed to a method of treating a skin condition preferably selected from wrinkles, fine lines, hyperpigmentation, uneven tone, loss of firmness, creepiness, surface roughness, dark circles, under-eye puffiness, crow's feet, visible sun damage, redness, dryness, irritation, skin sagging, skin slackening, enlarged pores and combinations thereof.

According to the present disclosure, the method of treating skin may further comprise the step of applying a topical skin care composition to the skin of the subject. A suitable topical skin care composition can be applied as frequently as desired, but preferably, though without limitation, at least once a day (once or twice a day), once a week, once a month, etc. A suitable topical skin care composition may take any form such as a cream, lotion, serum, facial cleanser, toner, eye cream, sunscreen, stick, spray, filled capsule, impregnated bandage, impregnated personal care device, impregnated towelette, gel, fluid/liquid, soap, transdermal patch, powder, liquid powder, cream powder, oil, butter, peel, scrub, mask, elixir, concentrate, capsule, semi-solid, or any other form known in the art. A singular suitable topical skin care composition may be used as part of the method of the present disclosure. Alternatively more than one suitable topical skin care compositions may be used as part of the method of the present disclosure, e.g., as part of a skin care regimen, i.e., several products used regularly in conjunction with one another; for example, a typical skin care regimen includes the use of a cleanser, toner, serum and eye cream twice daily, along with the use of a day cream once daily and a night cream once daily.

The steps of administering the oral supplement and applying the topical skin care composition can be carried out simultaneously or separately in any order.

A topical skin care composition preferred for use in the method of the present disclosure is that described in co-pending U.S. patent application Ser. No. ______ [filed concurrently herewith; entitled TOPICAL SKIN CARE COMPOSITION; attorney docket no. 04072.000100.]. The entire disclosure of this co-pending application is incorporated by reference herein. In some cases, combined use of the topical skin care compositions of the co-pending application (especially in a regimen as set forth above) and the oral supplement of the present disclosure will be found to accelerate skin hydration and reduce fine lines in half the time as compared to the use of the topical skin care compositions of the co-pending application only. In a preferred embodiment of the topical skin care composition of the co-pending application, the topical skin care composition comprises (a) an ascorbic acid source; (b) brown algae extract; (c) a blend of botanical extracts comprising cucumber extract, watercress extract, birch leaf extract, red clover extract, and St. John's wort extract; and (d) a cosmetically acceptable carrier. In further preferred embodiments, the blend of botanical extracts further comprises ginseng extract, the ascorbic acid source is selected from ascorbic acid, tetrahexyldecyl ascorbate, ascorbyl palmitate, magnesium ascorbyl phosphate, and combinations thereof, and the brown algae extract is obtained from a brown algae species selected from laminaria, ascophyllum, alaria, cladosiphan, durvillaea, ecklania, fucus, lessonia, macrocystis, sargassum, undoria, and combinations thereof.

Specific embodiments of the disclosure will now be demonstrated by reference to the following general methods of manufacture and examples. It should be understood that these examples are disclosed solely by way of illustration and should not be taken in any way to limit the scope of the present disclosure.

### Example 1

An oral supplement was prepared using the ingredients set forth in Table 2 below.

TABLE 2

| INGREDIENT | % w/w |
|---|---|
| Bilberry fruit extract | 5.49 |
| Sodium selenate | 0.29 |
| Quercetin dihydrate powder | 2.62 |
| Vitamin E (as mixed tocopherols) | 19.78 |
| Co-enzyme Q-10 powder | 3.53 |
| Biotin powder 1% | 3.63 |
| Ascorbic acid | 7.40 |
| Beta carotene beadlet | 3.29 |
| Vitamin A palmitate, vegetarian | 0.53 |
| Ergocalciferol | 0.60 |
| Zinc gluconate | 12.68 |
| Alpha-lipoic acid powder | 11.65 |
| Copper gluconate | 0.78 |
| Magnesium stearate EF | 1.10 |
| Silicon dioxide powder | 2.20 |
| Microcrystalline cellulose powder | 23.33 |
| Stearic acid powder | 1.10 |

The oral supplement was prepared by dry mixing the ingredients in Table 2 and then filling size B clear veggie capsules with 455 mg of the mixed ingredients.

### Example 2

The oral supplement of Example 1 was administered twice daily, i.e., in the morning (a.m.) and in the evening (p.m.), to a female subject. In the morning, the female subject also used a cleanser, toner, serum, eye cream and day cream as described in co-pending U.S. patent application Ser. No. ______ [filed concurrently herewith; entitled TOPICAL SKIN CARE COMPOSITION; attorney docket no. 04072.000100]. In the evening, the female subject also used a cleanser, toner, serum, eye cream and night cream as described in co-pending U.S. patent application Ser. No. ______ [filed concurrently herewith; entitled TOPICAL SKIN CARE COMPOSITION; attorney docket no. 04072.000100].

While the disclosure has been described above with reference to specific embodiments thereof, it is apparent that many changes, modifications, and variations can be made without departing from the concept disclosed herein. Accordingly, it is intended to embrace all such changes, modifications, and variations that fall within the spirit and broad scope of the appended claims.

What is claimed is:

1. An oral supplement comprising:
a) bilberry extract,
b) quercetin,
c) beta-carotene,
d) co-enzyme Q-10,
e) lipoic acid,
f) vitamin A,
g) vitamin B,
h) vitamin C,
i) vitamin D,
j) vitamin E
k) selenium,
l) zinc, and
m) copper,
wherein the oral supplement supports skin health.

2. The oral supplement according to claim 1 further comprising at least one inactive ingredient.

3. The oral supplement according to claim 2, wherein the at least one inactive ingredient is selected from the group consisting of magnesium stearate, silicon dioxide, microcrystalline cellulose, stearic acid, and combinations thereof.

4. The oral supplement according to claim 1, wherein the oral supplement is in the form of a powder-filled capsule.

5. A method of treating skin comprising the step of administering to a subject the oral supplement of claim 1 formulated to support skin health.

6. The method according to claim 5, wherein the oral supplement is administered at least once a day.

7. The method according to claim 5, wherein the oral supplement is administered in the form of at least one powder-filled capsule.

8. The method according to claim 5 further comprising at least one inactive ingredient.

9. The method according to claim 8, wherein the at least one inactive ingredient is selected from the group consisting of magnesium stearate, silicon dioxide, microcrystalline cellulose, stearic acid, and combinations thereof.

10. The method according to claim 5, wherein the subject suffers from a skin condition selected from the group consisting of wrinkles, fine lines, hyperpigmentation, uneven tone, loss of firmness, creepiness, surface roughness, dark circles, under-eye puffiness, crow's feet, visible sun damage, redness, dryness, irritation, skin sagging, skin slackening, enlarged pores and combinations thereof.

11. The method according to claim 5, further comprising the step of applying a topical skin care composition to the skin of the subject.

12. The method according to claim 11, wherein the topical skin care composition is applied at least once a day.

13. The method according to claim 12, wherein the topical skin care composition is applied twice a day.
